(19) United States
(12) Patent Application Publication
(10) Pub. No.: US 2018/0280337 A1
(43) Pub. Date: Oct. 4, 2018

(54) NUTRITIONAL COMPOSITIONS COMPRISING SN-1(3) MONOACYLGLYCEROLS FOR USE IN THE TREATMENT OF GROWTH DELAY IN INFANTS OR CHILDREN

(71) Applicant: NESTEC S.A., Vevey (CH)

(72) Inventors: Laurent AMEYE, Lausanne 26 (CH); Cristina CRUZ-HERNANDEZ, Epalinges (CH); Clara Lucia GARCIA-RODENAS, Forel (CH)

(21) Appl. No.: 15/522,087

(22) PCT Filed: Oct. 19, 2015
(86) PCT No.: PCT/EP2015/074113

§ 371 Date: Apr. 26, 2017

(30) Foreign Application Priority Data
Oct. 27, 2014 (EP) 14190532.3

(51) Int. Cl. A61K 31/232; A23L 33/12; A23L 33/00

(52) U.S. Cl. CPC A61K 31/232; A23V 2002/00; A23L 33/40; A23L 33/12

(57) ABSTRACT

The present invention refers to nutritional composition comprising sn-1(3) monoacylglycerols for use in the prevention/treatment of growth delay and/or in the promotion of growth in an infant or in a child like a young child, such as a preterm and/or low birth weight infants.

### FIELD OF THE INVENTION

The present invention relates generally to the field of lipids and in particular to nutritional compositions comprising sn-1(3) monoacylglycerols for use in the prevention/treatment of growth delay and/or in the promotion of growth in an infant or a child such as a young child. The sn-1(3) monoacylglycerols can comprise at least one functional fatty acid such as eicosapentaenoic acid (EPA), docosahexaenoic acid (DHA), eicosatetraenoic acid or arachidonic acid (ARA) for example. The present invention is also about the use of sn-1(3) monoacylglycerols in a nutritional composition to provide nutrition to an infant or a child such as a young child.

### BACKGROUND OF THE INVENTION

Lipids are normally consumed as triacylglycerols (TAG). During the digestion process, pancreatic lipases are secreted from the pancreas. Pancreatic triglyceride lipase (PTL) is the primary lipase that hydrolyzes dietary TAG molecules in the human digestive system to convert TAG to diacylglycerols (DAG) and ultimately to monoacylglycerols (MAG) and free fatty acids.

Bile salts secreted from the liver and stored in the gallbladder are released into the duodenum where they coat and emulsify large lipid droplets into smaller droplets, thus increasing the overall surface area of the lipid, which increases lipase efficiency. The resulting digestion products are then moved along the small intestine by peristalsis, waves of muscular contractions that move along the intestinal wall, to be absorbed into the enterocytes and transported by the lymphatic system. Although pancreatic lipases are secreted in their final active forms, they only become efficient in the presence of co-lipase in the duodenum.

The delivery of bioactive fatty acids under conditions of impaired lipid metabolism such as maldigestion, and under conditions of impaired uptake, known as malabsorption, is critical. These impairments contribute to malnutrition and specific nutrient deficits associated with reduced lipid assimilation. Additionally, the decrease in lipid absorption can cause steatorrhea, i.e. the presence of excess lipid in feces. This increases the likelihood of fecal incontinence and a strong offensive odor. Furthermore, the decrease in the absorption of saturated fatty acids, such as lauric, myristoleic, palmitic, stearic acids, can lead to the formation of complexes of the fatty acids with calcium. These complexes worsen the absorption rate of both fatty acids and calcium and lead to hard stools and impaired bone mineralization and growth.

The delivery of bioactive fatty acids having, e.g., anti-inflammatory properties, or of essential or conditionally essential fatty acids, especially important for cellular growth and functioning in key organs such as the brain or the eyes, is therefore critical in these conditions as this type of fatty acids could help to lower the inflammation response or to ensure a proper neurodevelopment.

The use of monoglyceride lipid instead of triglyceride lipid has been reported since the early 50's, both in animals, healthy volunteers and CF patients. Several publications (e.g. “Composition of intestinal lumen lipids following the feeding of triglycerides, partial glycerides or free fatty acids”, Matson F et al., J. Nutr. 1954; 52:575-79) indicate that lipids provided as monoglycerides are likely to give better absorption when compared to triglycerides.

Based on previously published prior art (Freeman C P, et al., J Dairy Sci 1965; 48:853-8; Innis S M, et al., Lipids 1994; 29:541-5) it is currently understood that fatty acids located in the sn-2 position of a glyceride are more readily absorbed by the body than fatty acids in the sn-1(3) position. One would hence assume that the provision of monoacylglycerols (MAGs) with a fatty acid in sn-2 position would be an ideal vehicle to provide fatty acids that can be easily absorbed. However, the use of sn-1(3) MAG is a preferred option since the technologies required to produce MAG from edible oils are well established and used for the production of emulsifiers, while the large scale production of sn-2 MAG derivatives will need to be developed having higher cost than sn-1(3) MAG.

WO2012/136659 describes compositions comprising a sn-1(3)-monoacylglycerol, wherein the acyl group is a fatty acid having anti-inflammatory properties for use in the treatment or prevention of inflammatory disorders, wherein the composition is to be administered to subjects suffering from a lipid maldigestion or malabsorption condition.

However this document is mainly focused on inflammatory diseases such as inflammatory bowel disease, Crohn's disease, chronic pancreatitis . . . . In addition, it refers only to subjects suffering from a lipid maldigestion or malabsorption condition due to a specific enzymatic deficiency or anatomic issue (e.g. pancreatic insufficiency, bile salt deficiency, short gut, cystic fibrosis, diabetes, pancreatic tumor, Shwachman-Diamond syndrome (SDS), chronic liver diseases, biliary fistula/obstruction, loss of absorptive surface, intestinal resection of bypass, small intestinal bacterial growth, defective enterocyte functions, lymphatic disorders, celiac disease, Zollinger-Ellison syndrome . . . ).

There is a need to develop compositions suitable for infants or children, especially infants and young children, taking into account that the infants and young children represent a specific sub-group of patients who have particular physiological conditions and require very specific needs. There is also a need to deliver compositions in a manner that does not involve a classical pharmaceutical intervention as the infants or young children are particularly fragile, in a manner that is easy of deliver, well accepted by the parents or health care practitioners, and that does keep the cost of such delivery reasonable and affordable by most.

The present inventors found that a nutritional composition comprising sn-1(3) monoacylglycerols could be particularly efficient for a specific sub-group of patients, namely infants and children, and particularly infants and young children. These patients have a limited capacity to digest lipids due to an immature digestive system and at the same time increased lipid demands to support organ growth and development. Nutritional compositions comprising sn-1(3) monoacylglycerols could therefore provide a particularly suitable nutrition to these infants/young children. They could especially be used in the prevention of growth delay and/or in the promotion of growth in an infant or a child such as a young child.

Such nutritional compositions could also be particularly effective in infants and children who are born preterm, i.e. before term, especially in infants and young children born preterm, more particularly preterm infants. They have usually a limited food intake and a specially impaired ability to digest fat. For the same reasons, the nutritional compositions of the present invention could be particularly effective on infants/children who have/had a low birth weight (i.e. low or very low or extremely low birth weight). The nutritional compositions of the present invention could also be particularly advantageous in other infants, young children or children at risk, such as those who are small for gestational age (SGA) and/or who are sick.

### SUMMARY OF THE INVENTION

The present invention deals about a nutritional composition comprising sn-1(3) monoacylglycerols for use in the prevention/treatment of growth delay and/or in the promotion of growth in an infant or a child, especially in an infant or young child.

In the present invention, the growth can refer to the size (i.e. height and/or weight) of said infant or young child. It can also refer to the size and/or development of any organ or tissue of said infant or child, especially infant or young child.

Interestingly, at least one of these specific benefits can also be obtained in an infant or a child (like a young child) receiving (or being administered, being given, feeding, eating, ingesting . . . ) the nutritional compositions according to the invention:
promotion of bone's growth and quality
cognitive development
motor and/or behavioural development
visual acuity development and/or reduction of ROP (retinopathy of prematurity) risks
lung development and/or reduction of BPD (bronchopulmonary dysplasia) risks
cardiovascular system health and development.

Another object of the invention is the use of sn-1(3) monoacylglycerols in a nutritional composition to provide nutrition to an infant or a child, especially an infant or young child.

The present invention is quite advantageous for infants and children at risk. The present invention is particularly advantageous for preterm infants/young children, even more particularly for preterm infants.

### FIGURES

FIG. 1 shows the chemical structure of a sn-1(3) MAG. R is a fatty acid (e.g. EPA . . . )

FIG. 2 shows the chemical structure of an example of EPA monoglycerides used in the present invention: sn-1(3)-monoeicosapentaenoylglycerol.

FIG. 3 shows the incorporation of EPA in erythrocytes resulting from treatments of control rats fed fish oil with or without XENICAL® (tetrahydrolipstatin) and rats fed with XENICAL® (tetrahydrolipstatin) with either vanillin acetal of 2-EPA (Group A), 1,3 diacetyl-2 EPA (Group B), and Sn-1(3) MAG-EPA (Group C). Values are means±SEM, n=6.

FIG. 4 shows the timeline of a clinical study supporting the concept of administering sn-1(3) MAG to promote absorption of fatty acids and fat-soluble nutrients in malabsorption or maldigestion conditions.

FIG. 5 shows acute effects in the clinical study, namely pharmacokinetic results as measured by EPA in chylomicrons, AUC over 10 hours postprandial.

FIG. 6 shows chronic effects in the clinical study, namely accretion of EPA in erythrocytes as percentage of total fatty acids after 21 days of treatment.

FIG. 7 shows chronic effects in the clinical study, namely accretion of EPA in plasma as percentage of total fatty acids after 21 days of treatment.

### DETAILED DESCRIPTION OF THE INVENTION

As used herein, the following terms have the following meanings.

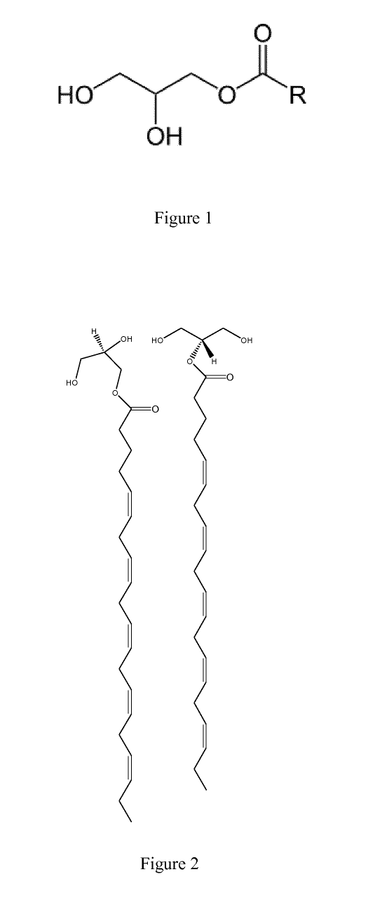

The expressions “sn-1(3) monoacylglycerol(s)” and “sn-1(3) MAG(s)” can be used interchangeably. They refer to fatty acid monoesters of glycerol wherein the sn-1 or sn-3 position is occupied by an acyl group such as a fatty acid and the sn-2 position remains unoccupied. A general structure is defined in FIG. 1.

The term “Infant” means a child (i.e. a young individual) under the age of 12 months.

The expression “young child” means a child (i.e. a young individual) aged between one and three years, also called toddler.

The term “child” encompasses a young individual aged between one and twelve years, especially between one and eight years, such as between one and six years or between one and five years, or between one and four years, or between three to eight years, or between three to six years or between three to five years. In some particular embodiments, the child is a young child.

A “preterm” or “premature” means an infant or a child who was not born at term. Generally it refers to an infant or a child who was born prior 37 weeks of gestation. The expressions “infant/young child/child born preterm”, “preterm infant/young child/child” and “preterms” can be used interchangeably.

A “term Infant/child” refers to an infant or a child born at term. Generally it refers to an infant or child who was born after 37 weeks of gestation.

An “Infant or child born by C-section” or an “Infant or child caesarian delivered” means an infant or child (such as a young child) who was delivered by caesarian (at the time of the birth). It means that the infant or child was not vaginally delivered.

An “Infant or child vaginally born” means an infant or a child (like a young child) who was vaginally delivered (at the time of the birth) and not delivered by caesarian for example.

By the expression “small for gestational age” or “SGA”, it is intended to mean an infant or child who is smaller in size than normal for the gestational age, most commonly defined as a weight below the 10th percentile for the gestational age. In some embodiments, SGA may be associated with IUGR (Intrauterine growth restriction), which refers to a condition in which a foetus is unable to achieve its genetically determined potential size.

By the expression “low birth weight”, it should be understood as any body weight under 2500 g at birth. It therefore encompasses:
infant or child who has/had a body weight from 1800 to 2500 g at birth (usually called “low birth weight” or LBW)
infant or child who has/had a body weight from 1000 to 1800 g at birth (called “very low birth weight” or VLBW)
infant or child who has/had a body weight under 1000 g at birth (called “extremely low birth weight” or ELBW)

In the present invention the “infant(s) at risk”, the “young child/children at risk” or the “child/children at risk” represent infant(s), young child/children or child/children having higher risks of developing a growth delay than usual (i.e. than the average), especially during the first month, 3 months, 6 months, 1 year, 2 years or 5 years of life, or even longer. This means that if we look at these infants, young children or children, there will be a higher incidence of growth delay, and/or a higher duration of the growth delay, and/or a higher severity of growth delay, and/or a longer time to relieve the symptoms of a growth delay in these infants, young children or children, in comparison with other infants, young children or children of the same age.

In a particular embodiment, the infant, young child or child at risk is an infant, a young child or a child who was born preterm and/or who is small for gestational age (SGA) and/or who has/had a low birth weight (i.e. low or very low or extremely low birth weight) and/or who is sick. In some embodiments it may be critically sick, i.e. with a life threatening illness or injury.

The expression “prevention/treatment of growth delay” encompasses the prevention of growth delay and/or the treatment of growth delay.

The expression “prevention of growth delay” in an infant or a child such as a young child means decreasing the incidence (reduction of the frequency) of growth delay and/or avoiding that growth delay occurs in said infant/child.

The term “treatment” does not necessarily imply that a subject is treated until total recovery.

The expression “treatment of growth delay” in an infant or a child such as a young child should be understood as comprising the decrease of growth delay (number of days/weeks/years the infants or children will suffer from growth delay) and/or the decrease of the severity of growth delay (the consequences and/or the seriousness of growth delay). This expression also encompasses the relieve of the symptoms of growth delay such as a low size (height and/or weight but in particular embodiments it refers to the height) of the infant/child, small or underdeveloped organs/tissues, and/or the decrease of complications caused by growth delay on the infant or child health, such as a compromised organ function, compromised cognitive, motor, emotional and social skills and an impaired socio-economic success.

The “growth delay” means that there is a lateness (i.e. a delay, a lower level . . . ) in the growth of said infant or child (like a young child) in comparison with the standard/average growth of other infants or children (like young children) of the same age. In some embodiments, the growth delay may be due to a birth before term. In some other embodiments, the growth delay may be due to physical or mental stress. In some other embodiments, the growth delay may be due to problems during pregnancy (e.g. insufficient nutrition by the mother, insufficient growth of the foetus such as IUGR). In some other embodiment, the growth delay may be due to some diseases/disorders leading for example to a fat maldigestion/malabsorption and/or to a limited food intake and/or to a reduced enteral feeding tolerance.

In some other embodiments, the growth delay may be due to a maldigestion or malabsorption of nutrients by the infant/child, such as a lipid (i.e. fat/fatty acid) maldigestion or malabsorption. The term “maldigestion” refers to the difficulty to digest (degrade) nutrients, for example due to a lack of key enzymes. The term “malabsorption” refers to the difficulty to absorb (integrate) the nutritional elements, for example due to a problem with the gut mucosa or as a consequence of an improper preliminary digestion.

In some particularly advantageous embodiments of the present invention, the maldigestion and/or the malabsorption refer to a lipid maldigestion and/or a lipid malabsorption. The term lipid means any fatty acids and/or any other fat molecules (designated by the term “fat”) including fat soluble nutrients like liposoluble vitamins and carotenoids. In a particular embodiment, the lipids are fatty acids, especially functional fatty acids.

The expression “lipid maldigestion” refers to an impaired lipid degradation in the gut lumen. The expression “lipid malabsorption” refers to an impaired lipid uptake through the gut mucosa.

The maldigestion and/or malabsorption may be due to a gut, a liver and/or an exocrine pancreas immaturity/insufficiency and/or to a gut inflammation and/or to a reduced gut mucosal surface and/or to an excessive gut motility and/or to a reduced enteral feeding tolerance of the infant/child.

The expression “promotion of growth” in an infant or child (such as young child) means that the growth of said infant/child is improved/enhanced while and/or after the administration of the nutritional composition of the invention. It also includes the promotion of catch-up growth for infants/child whose growth has been retarded due to physical or mental stress for example.

In any of these above-mentioned expressions, the term “growth” also encompasses the term “development”. It can refer to the development of the infant/child body, i.e. the (total) size (height and/or weight but in a particular embodiment it refers to the height) of the infant or of the child (such as a young child). It can alternatively refer to the development of any organ (lung, heart, eyes, ears, brain, gut, kidneys, reproductive organs, teeth, glands . . . ) and/or any tissue (bones, bone marrow, muscles, blood tissue, gland tissue, connective tissue, neural tissue . . . ) in said infant or child. In a particular embodiment of the invention, it encompasses both aspects.

In some embodiments, the prevention/treatment of growth delay and/or the promotion of growth may happen during the treatment (i.e. during the administration of the composition according to the present invention). It can also encompass the prevention/treatment or promotion later in life. The term “later in life” encompasses the effect after the termination of the intervention or treatment. The effect “later in life” can be from some days to several years, for example from 1 week to several months, for example from 2 to 4 weeks, from 2 to 6 weeks, from 2 to 8 weeks, from 1 to 6 months, from 2 to 12 months or from 3 to 36 months, or up to several years such as up to 2, 3, 5, 10, 15 or 18 years. The effect can be obtained after 1, 2, 3, 4, 5, 6, 7, 8, 9, 10, 11 or 12 months. It can also be obtained after 1, 2, 3, 4, 5, 6, 7, 8, 9, 10 or more years.

The term “weaning period” means the period during which the mother's milk is substituted by other food in the diet of an infant or a young child.

The expression “nutritional composition” means a composition which nourishes a subject. This nutritional composition is usually taken orally or intravenously, and it usually includes a lipid or fat source and a protein source. It also generally contains a carbohydrate source. In a particular embodiment, the nutritional composition contains only a lipid or fat source. In other specific embodiments, the nutritional composition contains a lipid (or fat) source with a protein source, a carbohydrate source or both. In a particular embodiment, the nutritional composition is not breast milk.

In some specific embodiments, the nutritional composition according to the invention is an “enteral nutritional composition” that is to say a foodstuff that involves the gastrointestinal tract for its administration. The infants or children may have no or a limited capacity to process oral foods: the gastric introduction will involve the use of a tube through the oro/nasal passage or a tube in the belly leading directly to the stomach. This may be used especially in hospitals or clinics.

The expression “hypoallergenic nutritional composition” means a nutritional composition which is unlikely to cause allergic reactions.

The expression “synthetic nutritional composition” means a mixture obtained by chemical and/or biological means, which can be chemically identical to the mixture naturally occurring in mammalian milks.

The expression “Infant formula” as used herein refers to a foodstuff intended for particular nutritional use by infants during the first months of life and satisfying by itself the nutritional requirements of this category of person (Article 2(c) of the European Commission Directive 91/321/EEC 2006/141/EC of 22 Dec. 2006 on infant formulae and follow-on formulae). It also refers to a nutritional composition intended for infants and as defined in Codex Alimentarius (Codex STAN 72-1981) and Infant Specialities (incl. Food for Special Medical Purpose). The infant formulas can encompass the starter infant formulas and the follow-up or follow-on formulas. Generally a starter formula is for infants from birth as breast-milk substitute. A follow-up or follow-on formula is given from the 6th month onwards. It constitutes the principal liquid element in the progressively diversified diet of this category of person.

The “growing-up milks” (or GUMs) are given from one year onwards. It is generally a milk-based beverage adapted for the specific nutritional needs of young children.

The expression “baby food” means a foodstuff intended for particular nutritional use by infants or children such as young children, during the first years of life.

The expression “Infant cereal composition” means a foodstuff intended for particular nutritional use by infants or children such as young children, during the first years of life.

The term “fortifier” refers to liquid or solid nutritional compositions suitable for mixing with breast milk (human milk) or infant formula. The “breast milk” should be understood as the mother's milk or the colostrum of the mother or a donor's milk or the colostrum of a donor's milk.

The term “supplement” may be used to complement the nutrition of an individual (it is typically used as such but it might also be added to any kind of compositions intended to be ingested). It may be in the form of tablets, capsules, pastilles or a liquid for example. The supplement may further contain protective hydrocolloids (such as gums, proteins, modified starches), binders, film forming agents, encapsulating agents/materials, wall/shell materials, matrix compounds, coatings, emulsifiers, surface active agents, solubilizing agents (oils, fats, waxes, lecithins etc.), adsorbents, carriers, fillers, co-compounds, dispersing agents, wetting agents, processing aids (solvents), flowing agents, taste masking agents, weighting agents, jellifying agents and gel forming agents. The supplement may also contain conventional pharmaceutical additives and adjuvants, excipients and diluents, including, but not limited to, water, gelatine of any origin, vegetable gums, lignin-sulfonate, talc, sugars, starch, gum arabic, vegetable oils, polyalkylene glycols, flavouring agents, preservatives, stabilizers, emulsifying agents, buffers, lubricants, colorants, wetting agents, fillers, and the like.

The term “HMO” or “HMOs” refers to human milk oligosaccharide(s). These carbohydrates are highly resistant to enzymatic hydrolysis, indicating that they may display essential functions not directly related to their caloric value. It has especially been illustrated that they play a vital role in the early development of infants and children like young children, such as the maturation of the immune system. Each individual oligosaccharide is based on a combination of glucose, galactose, sialic acid (N-acetylneuraminic acid), fucose and/or N-acetylglucosamine with many and varied linkages between them, thus accounting for the enormous number of different oligosaccharides in human milk—over 130 such structures have been identified so far. The HMOs can be acidic (e.g. charged sialic acid containing oligosaccharide) or neutral (e.g. fucosylated oligosaccharide).

The term “BMO” or “BMOs” refers to bovine milk oligosaccharides. The BMOs can be selected from the list comprising N-acetylated oligosaccharides, sialylated oligosaccharides and any mixtures thereof.

A “sialylated oligosaccharide” is a charged sialic acid containing oligosaccharide, i.e. an oligosaccharide having a sialic acid residue. It has an acidic nature. Some examples are 3-SL (3′ sialyllactose) and 6-SL (6′ sialyllactose). These sialylated oligosaccharides may be isolated by chromatographic or filtration technology from a natural source such as animal milks. Alternatively, they may also be produced by biotechnology using specific sialyltransferases either by enzyme based fermentation technology (recombinant or natural enzymes) or by microbial fermentation technology. In the latter case microbes may either express their natural enzymes and substrates or may be engineered to produce respective substrates and enzymes. Single microbial cultures or mixed cultures may be used. Sialyl-oligosaccharide formation can be initiated by acceptor substrates starting from any degree of polymerisation (DP) from DP=1 onwards.

A “fucosylated oligosaccharide” is an oligosaccharide having a fucose residue. It has a neutral nature. Some examples are 2-FL (2′-fucosyllactose), 3-FL (3-fucosyllactose), difucosyllactose, lacto-N-fucopentaose (e.g. lacto-N-fucopentaose I, lacto-N-fucopentaose II, lacto-N-fucopentaose III, lacto-N-fucopentaose V), lacto-N-fucohexaose, lacto-N-difucohexaose I, fucosyllacto-N-hexaose, fucosyllacto-N-neohexaose, difucosyllacto-N-hexaose I, difucosyllacto-N-neohexaose II and any combination thereof. Without wishing to be bound by theory it is believed that the fucosyl-epitope of the fucosylated oligosaccharides may act as decoy at the mucosal surface.

A “N-acetylated oligosaccharide” means an oligosaccharide having an N-acetyl residue

Suitable N-acetylated oligosaccharides include GalNAcα1,3Galβ1,4Glc and Galβ1,6GalNAcα1,3Galβ1,4Glc. The N-acetylated oligosaccharides may be prepared by the action of glucosaminidase and/or galactosaminidase on N-acetyl-glucose and/or N-acetyl galactose. Equally, N-acetyl-galactosyl transferases and/or N-acetyl-glycosyl transferases may be used for this purpose. The N-acetylated oligosaccharides may also be produced by fermentation technology using respective enzymes (recombinant or natural) and/or microbial fermentation. In the latter case the microbes may either express their natural enzymes and substrates or may be engineered to produce respective substrates and enzymes. Single microbial cultures or mixed cultures may be used. N-acetylated oligosaccharide formation can be initiated by acceptor substrates starting from any degree of polymerisation (DP) from DP=1 onwards. Another option is the chemical conversion of keto-hexoses (e.g. fructose) either free or bound to an oligosaccharide (e.g. lactulose) into N-acetylhexosamine or an N-acetylhexosamine containing oligosaccharide as described in Wrodnigg, T. M.; Stutz, A. E. (1999) Angew. Chem. Int. Ed. 38:827-828.

A “galacto-oligosaccharide” is typically an oligosaccharide comprising two or more galactose molecules which has no charge and no N-acetyl residue. In a particular embodiment, it may also be a GOS disaccharide composed of one Gal and one Glc. Suitable galacto-oligosaccharides include Galβ1,6Gal, Galβ1,6Galβ1,4Glc Galβ1,6Galβ1,6Glc, Galβ1,3Galβ1,3Glc, Galβ1,3Galβ1,4Glc, Galβ1,6Galβ1,6Galβ1,4Glc, Galβ1,6Galβ1,3Galβ1,4Glc Galβ1,3Galβ1,6Galβ1,4Glc, Galβ1,3Galβ1,3Galβ1,4Glc, Galβ1,4Galβ1,4Glc and Galβ1,4Galβ1,4Galβ1,4Glc. Synthesised galacto-oligosaccharides such as Galβ1,6Galβ1,4Glc Galβ1,6Galβ1,6Glc, Galβ1,3Galβ1,4Glc, Galβ1,6Galβ1,6Galβ1,4Glc, Galβ1,6Galβ1,3Galβ1,4Glc and Galβ1,3Galβ1,6Galβ1,4Glc, Galβ1,4Galβ1,4Glc and Galβ1,4Galβ1,4Galβ1,4Glc and mixtures thereof are commercially available under the trademarks Vivinal® and Elix′ or ®. Other suppliers of oligosaccharides are Dextra Laboratories, Sigma-Aldrich Chemie GmbH and Kyowa Hakko Kogyo Co., Ltd. Alternatively, specific glycoslytransferases, such as galactosyltransferases may be used to produce neutral oligosaccharides.

The term “preblotic” means non-digestible carbohydrates that beneficially affect the host by selectively stimulating the growth and/or the activity of healthy bacteria such as bifidobacteria in the colon of humans (Gibson G R, Roberfroid M B. Dietary modulation of the human colonic microbiota: introducing the concept of prebiotics. J Nutr. 1995; 125:1401-12).

The term “probiotic” means microbial cell preparations or components of microbial cells or products of microbial metabolism having a beneficial effect on the health or well-being of the host. (Salminen S, Ouwehand A. Benno Y. et al. “Probiotics: how should they be defined” Trends Food Sci. Technol. 1999:10 107-10). The microbial cells are generally bacteria or yeasts.

The term “cfu” should be understood as colony-forming unit.

All percentages are by weight unless otherwise stated. The expressions “weight %” and “wt %/o” are synonymous. They refer to quantities expressed in percent on a dry weight basis.

The invention will now be described in further details. It is noted that the various aspects, features, examples and embodiments described in the present application may be compatible and/or combined together.

In addition, in the context of the invention, the terms “comprising” or “comprises” do not exclude other possible elements. The composition of the present invention, including the many embodiments described herein, can comprise, consist of, or consist essentially of the essential elements and limitations of the invention described herein, as well as any additional or optional ingredients, components, or limitations described herein or otherwise depending on the needs.

An object of the present invention is a nutritional composition comprising sn-1(3) monoacylglycerols for use in the prevention/treatment of growth delay and/or in the promotion of growth in an infant or a child, especially in an infant or a young child.

Another object of the present invention refers to the use of sn-1(3) monoacylglycerols in a nutritional composition to provide nutrition (or a suitable nutrition, i.e. a feed that fulfils all the requirements/needs of an individual) to an infant or a child, especially to an infant or young child.

The sn-1(3) monoacylglycerols (sn-1(3) MAGs) were found to be effective glyceride structures allowing a substantial uptake of fatty acids—such as DHA or EPA—more effectively than fish oil.

MAGs do not need to be digested prior to absorption and have intrinsic emulsifying properties allowing a good dispersion of oil droplets prior to absorption in the intestine.

The inventors tested their concept in lipid maldigestion/malabsorption animal and human models. The malabsorption condition was obtained using XENICAL® (Orlistat), a well-known pancreatic and gastric lipases inhibitor. As detailed in the examples, the level of EPA incorporated in blood cells both in animals and humans receiving a nutritional composition comprising sn-1(3)MAGs was found to be significantly higher compared to the administration of fish oil. This clearly demonstrates that if LC-PUFAs are provided as sn-1(3) MAGs, the incorporation of EPA in tissue is enhanced, also in conditions of lipid malabsorption/maldigestion.

Typically the sn-1(3)-monoacylglycerol of the nutritional composition according to the invention may be selected from the group consisting of:
sn-1(3)-monohexadecanoylglycerol,
sn-1(3)-monotetradecanoylglycerol,
sn-1(3)-monooctadecanoylglycerol,
sn-1(3)-monooctadecadienoylglycerol,
sn-1(3)-monoeicosatetraenoylglycerol,
sn-1(3)-monoeicosapentaenoylglycerol,
sn-1(3)-monodocosahexaenoylglycerol,
sn-1(3)-monooctadecatrienoylglycerol,
sn-1(3)-monooctadecatetraenoylglycerol,
sn-1(3)-monoeicosatrienoylglycerol,
sn-1(3)-monodocosapentaenoylglycerol,
sn-1(3)-monosciadonylglycerol,
sn-1(3)-monojuniperonylglycerol,
or any combinations thereof.

In some advantageous embodiments, the sn-1(3)-monoacylglycerol is selected from the group consisting of sn-1(3)-monoeicosatrienoylglycerol, sn-1(3)-monodocosahexaenoylglycerol, sn-1(3)-monoeicosatetraenoylglycerol, sn-1(3)-monooctadecatrienoylglycerol, sn-1(3)-monooctadecadienoylglycerol and sn-1(3)-monoeicosapentaenoylglycerol.

In a particular embodiment, the sn-1(3)-monoacylglycerol is sn-1(3) monoeicosapentaenoylglycerol.

In a particularly advantageous embodiment, the sn-1(3)-monoacylglycerol is mono docosahexaenoylglycerol.

In a particular embodiment, the sn-1(3)-monoacylglycerol is sn-1(3)-monoeicosatetraenoylglycerol.

In preferred embodiments, the sn-1(3) monoacylglycerols comprises at least one functional fatty acid. It is typically the acyl group of the sn-1(3) monoacylglycerols that may be a functional fatty acid.

A functional fatty acid is a fatty acid that is key for survival and/or provides a health benefit to an individual administered the fatty acid. A functional fatty acid can be an essential, a conditionally essential and/or a bioactive fatty acid. An essential fatty acid is a fatty acid that cannot be synthesized by the body and therefore needs to be provided by the diet. A conditionally essential fatty acid is a fatty acid that can be synthesized by the body, but which, in particular circumstances such as fast growth or disease is required in amounts larger than those synthesised by the body. In these particular circumstances, the conditionally essential fatty acid needs to be provided by the diet. A bioactive fatty acid is a fatty acid that might not be essential for survival but which provision in the diet leads to a specific health benefit for example cellular growth or functioning of key organs such as the brain or the eyes.

Non-limiting examples of functional fatty acids include tetradecanoic acid (myristic acid), hexadecanoic acid (palmitic acid), octadecanoic acid (stearic acid), eicosapentaenoic acid (EPA), docosahexaenoic acid (DHA), alpha-linolenic acid (ALA), linoleic acid (LA), conjugated linoleic acid (CLA), arachidonic acid (ARA), stearidonic acid (SA), γ-linolenic acid (GLA), dihomo-γ-linolenic acid (DGLA), n-3 docosapentanenoic acid (DPA), sciadonic acid and juniperonic acid.

Sciadonic acid may be 5Z, 11Z, 14Z-eicosatrienoic acid.

Juniperonic acid may be 5(Z), 11(Z), 14(Z), 17(Z)-eicosatetraenoic acid.

In some advantageous embodiments of the invention, the functional fatty acid is EPA, DHA, ARA, LA and/or ALA. In some advantageous embodiments of the invention, the functional fatty acid is DHA, ARA, LA and/or ALA. In some advantageous embodiments of the invention, the functional fatty acid is EPA, DHA and/or ARA. In a specific embodiment, it is EPA. In another particular embodiment, it is DHA. In another embodiment, it is ARA. In another embodiment, it is LA. In another embodiment, it is ALA.

In some particular advantageous embodiments of the invention, the sn-1(3) monoacylglycerols (MAG) therefore comprise at least one of sn-1(3) MAG-EPA, sn-1(3) MAG-DHA, sn-1(3) MAG-ARA, sn-1(3) MAG-LA or sn-1(3) MAG-ALA.

In some particular advantageous embodiments of the invention, the sn-1(3) monoacylglycerols (MAG) therefore comprise at least one of sn-1(3) MAG-DHA, sn-1(3) MAG-ARA, sn-1(3) MAG-LA or sn-1(3) MAG-ALA.

In some particular advantageous embodiments of the invention, the sn-1(3) monoacylglycerols (MAG) therefore comprise at least one of sn-1(3) MAG-EPA, sn-1(3) MAG-DHA or sn-1(3) MAG-ARA.

The nutritional composition of the invention may comprise a mixture of different sn-1(3) MAGs, e.g. sn-1(3) MAGs with different fatty acids in the sn-1(3) position.

The fatty acids may be mixed in a way, for example, that a particular ratio between n-3 and n-6 fatty acids is used.

Non-limiting examples of suitable n-3 fatty acids include for example α-linolenic acid, stearidonic acid, eicosatrienoic acid, n-3 eicosatetraenoic acid, eicosapentaenoic acid, clupanodonic acid, docosahexaenoic acid, n-3 tetracosapentaenoic acid or n-3 tetracosahexaenoic acid.

Non-limiting examples of suitable n-6 fatty acids include for example linoleic acid, γ-linolenic acid, n-6 eicosadienoic acid, dihomo-γ-linolenic acid, arachidonic acid, n-6 docosadienoic acid, adrenic acid, n-6 docosapentaenoic acid or calendic acid.

The nutritional composition may contain a combination of different sn-1(3) monoacylglycerides; for example with a ratio of n-3 to n-6 fatty acids of about 5:1 to about 15:1; for example about 8:1 to about 10:1.

Optionally, the composition contains sn-2 MAG in addition to the sn-1(3) MAG. The nutritional composition of the present invention may therefore comprise a mixture of sn-2 MAGs and sn-1(3) MAGs.

Depending on the nature of the fatty acid used as acyl-group in the sn-1(3) position, such mixtures may form automatically through isomerization. Therefore, in an embodiment of the present invention, the nutritional composition comprises 25% or less by weight of the total MAG as sn-2 MAG, preferably 15% or less by weight of the total MAG as sn-2 MAG. The sn-1 and sn-3 positions of the sn-2 MAG can be blocked by protective groups to limit isomerization. Non-limiting examples of suitable protective groups include acetyl groups, ethyl groups, propyl groups, vanillin, and other molecules able to form acetals. In some embodiments, the protective group bridges the hydroxyl groups in sn-1 and sn-3 positions. Unwanted isomerisation may also be prevented or at least slowed down significantly by adjusting the pH to the neutral range and/or by keeping the temperature of the composition low. Hence, the nutritional composition may have a pH in the range of about 5-8, such as about 5-7.

The nutritional composition may also be to be stored at 8° C. or below.

Non-limiting examples of suitable sn-2 MAG include:
1,3-diacetyl-2-eicosapentaenoylglycerol
1,3-diacetyl-2-docosahexaenoylglycerol
1,3-diacetyl-2-eicosatetraenoylglycerol acid
1,3-diacetyl-2-eicosatrienoylglycerol 1,3-diethyl-2-eicosapentaenoylglycerol
1,3-diethyl-2-docosahexaenoylglycerol
1,3-diethyl-2-eicosatrienoylglycerol
1,3-dipropyl-2-eicosapentaenoylglycerol
1,3-dipropyl-2-docosahexaenoylglycerol
1,3-dipropyl-2-eicosatrienoylglycerol
a vanillin derivative of sn-2 monoeicosapentaenoylglycerol
sn-2 monodocosahexaenoylglycerol
sn-2-monoeicosatetraenoylglycerol acid
sn-2 monoeicosatrienoylglycerol
other acetal derivatives of monoeicosapentaenoylglycerol, of monodocosahexaenoylglycerol or of monoeicosatrienoylglycerol
or any combinations thereof.

Hence, the nutritional composition of the present invention allows delivering functional bioactive fatty acids such EPA, DHA and/or ARA in infants or children (e.g. young children) in a more bioavailable form for the body. It also allows preventing the complexation of certain fatty acids such as palmitic acid with calcium.

The nutritional composition of the present invention is thought to be very effective in the prevention/treatment of growth delay and/or in the promotion of growth in an infant or a child, especially in an infant or a young child. It will also provide or a suitable nutrition to an infant or to a child (such as a young child).

Using sn-1(3) monoacylglycerides as suitable vehicles to efficiently deliver functional (e.g. bioactive) fatty acids would therefore be particularly advantageous for said infants/children. The fat/fatty acids absorption will be improved in said infants/children. Functional fatty acids are key for the development of the infants/children. A decrease of growth delay and/or a promotion of growth can be expected in the infants/children ingesting the nutritional composition according to the invention.

In addition, infants and children, and more particularly infants and young children represent a specific sub-group of individuals having often a gut immaturity and/or a reduced enteral feeding tolerance in comparison with other types of individuals (e.g. adults). Infants and children have also particularly high requirements in fat and functional fatty acids due to the needs for growth and organ development. The need of a nutritional composition comprising a vehicle delivering functional fatty acids is therefore increased in this sub-population.

The growth may refer to the body (i.e. the size, that may be the height and/or the weight but in particular embodiments it refers to the height) of said infant or of the child (like young child) and/or to any organ or tissue development of said infant or child (like a young child). Non-limiting examples of organs are lung, heart, eyes, ears, brain, gut, kidneys, reproductive organs, teeth, glands . . . . Non-limiting examples of tissues are bones, bone marrow, muscles, blood tissue, gland tissue, connective tissue, neural tissue.

Therefore in one embodiment, the nutritional composition according to the present invention is for use in the prevention/treatment of growth (i.e. development) delay of the body of the infant or of the child, especially of the infant or of the young child.

In another embodiment, the nutritional composition according to the present invention is for use in the prevention/treatment of growth (i.e. development) delay of any organ or tissue development of said infant or child, especially of said infant or young child.

In another embodiment, the nutritional composition according to the present invention is for use in the promotion of growth (i.e. development) of the body of the infant or child, especially of the infant or young child.

In another embodiment, the nutritional composition according to the present invention is for use in the promotion of growth (i.e. development) of any organ or tissue development of said infant or child, especially of said infant or young child.

Interestingly, at least one of these specific benefits can be obtained in the infant or the child (like a young child) receiving (or administering, feeding, eating, ingesting, given . . . ) the nutritional composition according to the invention:
promotion of bone's growth and quality
cognitive development
motor and/or behavioural development
visual acuity development and/or reduction of ROP (retinopathy of prematurity) risks
lung development and/or reduction of BPD (bronchopulmonary dysplasia) risks
cardiovascular system health and development.

Other advantages may also be obtained in said infants or children (like young children) such as the development of a healthier microbiota, a reduction of inflammation, a reduction of the risks of sepsis, a reduction of the risk of allergy.

As previously mentioned, the nutritional composition of the present invention is thought to be particularly effective in an infant or a young child. In a preferred embodiment, the nutritional composition of the invention is for use in infants.

The nutritional composition according to the invention can be used in infants or children who were born at term or preterm (i.e. either term infants/children or preterm infants/children). In some embodiments, the nutritional composition is used in infants or young children who were born at term or preterm (i.e. either term infants/young children or preterm infants/young children).

In a particularly advantageous embodiment, the nutritional composition of the invention is for use in infants or young children who were born preterm (i.e. preterm infants/young children). In a particularly advantageous embodiment the nutritional composition of the invention is for use in preterm infants.

In some embodiments of the invention, the nutritional composition can be used in infants or children (like young children) who were vaginally delivered. In some embodiments of the invention, the nutritional composition can be used in infants or children (like young children) who were caesarian delivered.

In some particular embodiments, the infant or the child (e.g. a young child) is an infant or a child at risk. In a particular embodiment, the infant or child at risk is an infant or a child born preterm and/or who is small for gestational age (SGA) and/or who has/had a low birth weight (i.e. low or very low or extremely low birth weight) and/or who is sick, such as critically ill (i.e. who has a life threatening illness or injury).

In a particular embodiment, the nutritional composition is for use in an infant or a child (such as a young child) born preterm and/or who is small for gestational age (SGA) and/or who has/had a low birth weight.

In a particular embodiment, the nutritional composition is for use in an infant or a child (such as a young child) born preterm and who is small for gestational age (SGA) and/or who has/had a low birth weight.

In a particular embodiment, the nutritional composition is for use in an infant or a child (such as a young child) born preterm.

In another embodiment, the nutritional composition is for use in small for gestational age (SGA) infant/child.

In another embodiment, the infant or child is low birth weight (i.e. low or very low or extremely low birth weight).

In a particular embodiment, the nutritional composition is for use in an infant/child born preterm and small for gestational age (SGA).

The nutritional composition of the invention is thought to be particularly effective in infants and children born preterm, especially in infants and young children born preterm, i.e. before term. Therefore in a preferred embodiment, the nutritional composition of the invention is for use in preterm infants/young children, preferably in preterm infants. Indeed, the fat digestion/absorption is impaired in preterms compared to term infants due to gut immaturity and co-morbidities. The volume of food and therefore the amount of fat that preterms can ingest is limited due to reduced tolerance to enteral feeding. In addition, the stores of fat (thus of the different fatty acids) at birth are much lower in preterm than in term infants. The metabolic processes allowing synthesis of certain fatty acids (e.g. DHA) by the body are immature (impaired) in preterms. Moreover the growth rate and, therefore, the fatty acid accretion in different tissues (e.g. DHA into brain and retina, fat into the adipose tissue) is potentially faster in preterm than in term infants.

Therefore the requirements in certain fatty acids (e.g. ALA, LA, DHA, AA) can be higher in preterm than in term infants. An increase in the dose is not always possible due to technological problems (e.g. high sensitivity to oxidation which introduces burden in product elaboration and shortens shelf life; poor organoleptic properties). The use of sn-1(3) monoacylglycerols as vehicle would therefore allow to more efficiently deliver the required amount of these fatty acids.

For the same reasons, the nutritional compositions of the present invention could be particularly effective on infants/young children who have/had a low birth weight (i.e. low or very low or extremely low birth weight).

In some particular embodiments, the nutritional compositions of the present invention is for use in infants/young children born preterm and who have/had a low birth weight (i.e. low or very low or extremely low birth weight).

It should be pointed out that the present invention could also be perfectly applied on young pets and/or young mammals, especially young dogs and young cats.

The nutritional composition according to the invention can be for example a synthetic nutritional composition. It can be an infant formula (e.g. a starter infant formula, a follow-up or a follow-on formula), a growing-up milk, a baby food, an infant cereal composition, a fortifier such as a human milk fortifier, or a supplement. In some particular embodiments, the nutritional composition of the invention is an infant formula, a fortifier or a supplement intended for the first 4 to 6 months of age of the infant.

In a specific embodiment the nutritional composition according to the invention is an enteral nutritional composition.

In a particular embodiment the nutritional composition of the present invention is an infant formula.

In another particular embodiment the nutritional composition of the present invention is a fortifier. The fortifier can be a breast milk fortifier or a formula fortifier such as an infant formula fortifier. The fortifier is therefore a particularly advantageous embodiment when the infant or child (like a young child) is born preterm.

When the nutritional composition is a supplement, it can be provided in the form of unit doses.

In some embodiments the nutritional composition according to the invention can be for use before and/or during the weaning period.

The nutritional composition of the present invention can be in solid (e.g. powder), liquid or gelatinous form.

For example, when the infant or child (like a young child) is born low birth weight or preterm, the nutritional composition could advantageously be a nutritional composition consumed in liquid form. In this case it may be a nutritionally complete formula such as an infant formula or a fortifier such as a human milk fortifier.

The nutritional composition of the invention, and especially the infant formula, generally contains a protein source, a carbohydrate source and a lipid source.

In some embodiments however, especially if the nutritional composition of the invention is a supplement or a fortifier, there may be only lipids (or a lipid source). In a particular embodiment, the composition will contain (consist of) only sn-1(3) monoacylglycerols as defined in the present invention. In some other embodiments, the nutritional composition of the invention may comprise a lipid source with a protein source, a carbohydrate source or both.

As already explained, the nutritional composition according to the present invention contains a source of lipids comprising the sn-1(3) monoacylglycerols as defined in the present invention. Other lipids may be present in addition to the sn-1(3) monoacylglycerols. The lipid source may be any lipid or fat which is suitable for use in infant formula for example. Some suitable fat sources include palm oil, high oleic sunflower oil, coconut oil, milk fat and/or high oleic safflower oil. The essential fatty acids linoleic and α-linolenic acid may also be added, as well small amounts of oils containing high quantities of preformed arachidonic acid and docosahexaenoic acid such as fish oils or microbial oils. The fat source may have a ratio of n-6 to n-3 fatty acids of about 5:1 to about 15:1; for example about 8:1 to about 10:1.

The nutritional composition according to the invention generally contains a protein source. The protein can be in an amount of from 1.6 to 3 g per 100 kcal. In some embodiments, especially when the composition is intended for preterm infants/young children, the protein amount can be between 2.4 and 4 g/100 kcal or more than 3.6 g/100 kcal. In some other embodiments the protein amount can be below 2.0 g per 100 kcal, e.g. between 1.8 to 2 g/100 kcal, or in an amount below 1.8 g per 100 kcal.

The type of protein is not believed to be critical to the present invention provided that the minimum requirements for essential amino acid content are met and satisfactory growth is ensured. Thus, protein sources based on whey, casein and mixtures thereof may be used as well as protein sources based on soy. As far as whey proteins are concerned, the protein source may be based on acid whey or sweet whey or mixtures thereof and may include alpha-lactalbumin and beta-lactoglobulin in any desired proportions.

In some advantageous embodiments the protein source is whey predominant (i.e. more than 50% of proteins are coming from whey proteins, such as 60% or 70%).

The proteins may be intact or hydrolysed or a mixture of intact and hydrolysed proteins. By the term “intact” is meant that the main part of the proteins are intact, i.e. the molecular structure is not altered, for example at least 80% of the proteins are not altered, such as at least 85% of the proteins are not altered, preferably at least 90% of the proteins are not altered, even more preferably at least 95% of the proteins are not altered, such as at least 98% of the proteins are not altered. In a particular embodiment, 100% of the proteins are not altered.

The term “hydrolysed” means in the context of the present invention a protein which has been hydrolysed or broken down into its component amino acids.

The proteins may be either fully or partially hydrolysed. It may be desirable to supply partially hydrolysed proteins (e.g. with a degree of hydrolysis between 2 and 20%), for example for infants or children believed to be at risk of developing cow's milk allergy. If hydrolysed proteins are required, the hydrolysis process may be carried out as desired and as is known in the art. For example, whey protein hydrolysates may be prepared by enzymatically hydrolysing the whey fraction in one or more steps. If the whey fraction used as the starting material is substantially lactose free, it is found that the protein suffers much less lysine blockage during the hydrolysis process. This enables the extent of lysine blockage to be reduced from about 15% by weight of total lysine to less than about 10% by weight of lysine; for example about 7% by weight of lysine which greatly improves the nutritional quality of the protein source.

In an embodiment of the invention at least 70% of the proteins are hydrolysed, preferably at least 80% of the proteins are hydrolysed, such as at least 85% of the proteins are hydrolysed, even more preferably at least 90% of the proteins are hydrolysed, such as at least 95% of the proteins are hydrolysed, particularly at least 98% of the proteins are hydrolysed. In a particular embodiment, 100% of the proteins are hydrolysed.

In one particular embodiment the proteins of the composition are hydrolysed, fully hydrolysed or partially hydrolysed. The degree of hydrolysis (DH) of the protein can be between 2 and 20, or between 8 and 40, or between 20 and 60 or between 20 and 80 or more than 10, 20, 40, 60, 80 or 90. For example, nutritional compositions containing hydrolysates having a degree of hydrolysis less than about 15% are commercially available from Nestle Company under the trade mark Peptamen®. Hydrolysates having a degree of hydrolysis above about 15% may be prepared using the procedure described in EP 0322589.

In a particular embodiment the nutritional composition according to the invention is a hypoallergenic nutritional composition.

The nutritional composition according to the present invention generally contains a carbohydrate source. This is particularly preferable in the case where the nutritional composition of the invention is an infant formula. In this case, any carbohydrate source conventionally found in infant formulae such as lactose, sucrose, saccharose, maltodextrin, starch and mixtures thereof may be used although one of the preferred sources of carbohydrates is lactose.

The nutritional composition of the invention may also contain all vitamins and minerals understood to be essential in the daily diet and in nutritionally significant amounts. Minimum requirements have been established for certain vitamins and minerals. Examples of minerals, vitamins and other nutrients optionally present in the composition of the invention include vitamin A, vitamin B1, vitamin B2, vitamin B6, vitamin B12, vitamin E, vitamin K, vitamin C, vitamin D, folic acid, inositol, niacin, biotin, pantothenic acid, choline, calcium, phosphorous, iodine, iron, magnesium, copper, zinc, manganese, chlorine, potassium, sodium, selenium, chromium, molybdenum, taurine, and L-carnitine. Minerals are usually added in salt form. The presence and amounts of specific minerals and other vitamins will vary depending on the intended population.

If necessary, the nutritional composition of the invention may contain emulsifiers and stabilisers such as soy, lecithin, citric acid esters of mono- and diglycerides, and the like.

The nutritional composition of the invention may also contain other substances which may have a beneficial effect such as lactoferrin, osteopontin, TGFbeta, sIgA, glutamine, nucleotides, nucleosides, and the like.

The nutritional composition of the invention can further comprise at least one non-digestible oligosaccharide (e.g. prebiotics). They are usually in an amount between 0.3 and 10% by weight of composition.

Prebiotics are usually non-digestible in the sense that they are not broken down and absorbed in the stomach or small intestine and thus remain intact when they pass into the colon where they are selectively fermented by the beneficial bacteria. Examples of prebiotics include certain oligosaccharides, such a fructooligosaccharides (FOS), inulin, xylooligosaccharides (XOS), polydextrose or any mixture thereof. In a particular embodiment, the prebiotics may be fructooligosaccharides and/or inulin. In a specific embodiment, the prebiotics is a combination of FOS with inulin such as in the product sold by BENEO-Orafti under the trademark Orafti® oligofructose (previously Raftilose®) or in the product sold by BENEO-Orafti under the trademark Orafti® inulin (previously Raftiline®). Another example is a combination of 70% short chain fructooligosaccharides and 30% inulin, which is registered by Nestle under the trademark “Prebio 1”.

The nutritional composition of the invention can also comprise at least one milk's oligosaccharide that can be a BMO (bovine milk oligosaccharide) and/or a HMO (human milk oligosaccharide), as previously detailed. In a particular embodiment, the nutritional composition according to the invention comprises an oligosaccharide mixture comprising from 0.1 to 4.0 wt % of N-acetylated oligosaccharide(s), from 92.0 to 98.5 wt % of the galacto-oligosaccharide(s) and from 0.3 to 4.0 wt % of the sialylated oligosaccharide(s).

The nutritional composition of the present invention can further comprise at least one probiotic (or probiotic strain), such as a probiotic bacterial strain.

The probiotic microorganisms most commonly used are principally bacteria and yeasts of the following genera: Lactobacillus spp., Streptococcus spp., Enterococcus spp., Bifidobacterium spp. and Saccharomyces spp.

In some particular embodiments, the probiotic is a probiotic bacterial strain. In some specific embodiments, it is particularly Bifidobacteria and/or Lactobacilli.

Suitable probiotic bacterial strains include Lactobacillus rhamnosus ATCC 53103 available from Valio Oy of Finland under the trademark LGG, Lactobacillus rhamnosus CGMCC 1.3724, Lactobacillus paracasei CNCM I-2116, Lactobacillus johnsonii CNCM I-1225, Streptococcus salivarius DSM 13084 sold by BLIS Technologies Limited of New Zealand under the designation K12, Bifidobacterium lactis CNCM 1-3446 sold inter alia by the Christian Hansen company of Denmark under the trademark Bb 12, Bifidobacterium longum ATCC BAA-999 sold by Morinaga Milk Industry Co. Ltd. of Japan under the trademark BB536, Bifidobacterium breve sold by Danisco under the trademark Bb-03, Bifidobacterium breve sold by Morinaga under the trade mark M-16V, Bifidobacterium infantis sold by Procter & Gamble Co. under the trademark Bifantis and Bifidobacterium breve sold by Institut Rosell (Lallemand) under the trademark R0070.

The nutritional composition according to the invention typically contains from 10e3 to 10e12 cfu of probiotic strain, more preferably between 10e7 and 10e12 cfu such as between 10e8 and 10e10 cfu of probiotic strain per g of composition on a dry weight basis.

In one embodiment the probiotics are viable. In another embodiment the probiotics are non-replicating or inactivated. It may also be probiotic parts such as cell wall components or products of the probiotic metabolism. There may be both viable probiotics and inactivated probiotics in some other embodiments.

The nutritional composition of the invention can further comprise at least one phage (bacteriophage) or a mixture of phages, preferably directed against pathogenic Streptococci, Haemophilus, Moraxella and Staphylococci.

The nutritional composition according to the invention may be prepared in any suitable manner. A composition will now be described by way of example.

For example, a formula such as an infant formula may be prepared by blending together the protein source, the carbohydrate source and the fat source comprising the sn-1(3) monoacylglycerols as defined in the present invention, in appropriate proportions. If used, the emulsifiers may be included at this point. The vitamins and minerals may be added at this point but they are usually added later to avoid thermal degradation. Any lipophilic vitamins, emulsifiers and the like may be dissolved into the fat source prior to blending. Water, preferably water which has been subjected to reverse osmosis, may then be mixed in to form a liquid mixture. The temperature of the water is conveniently in the range between about 50° C. and about 80° C. to aid dispersal of the ingredients.

Commercially available liquefiers may be used to form the liquid mixture.

Any oligosaccharides may be added at this stage, especially if the final product is to have a liquid form. If the final product is to be a powder, they may likewise be added at this stage if desired.

The liquid mixture is then homogenised, for example in two stages.

The liquid mixture may then be thermally treated to reduce bacterial loads, by rapidly heating the liquid mixture to a temperature in the range between about 80° C. and about 150° C. for a duration between about 5 seconds and about 5 minutes, for example. This may be carried out by means of steam injection, an autoclave or a heat exchanger, for example a plate heat exchanger.

Then, the liquid mixture may be cooled to between about 60° C. and about 85° C. for example by flash cooling. The liquid mixture may then be again homogenised, for example in two stages between about 10 MPa and about 30 MPa in the first stage and between about 2 MPa and about 10 MPa in the second stage. The homogenised mixture may then be further cooled to add any heat sensitive components, such as vitamins and minerals. The pH and solids content of the homogenised mixture are conveniently adjusted at this point.

If the final product is to be a powder, the homogenised mixture is transferred to a suitable drying apparatus such as a spray dryer or freeze dryer and converted to powder. The powder should have a moisture content of less than about 5% by weight. Any oligosaccharides may also be added at this stage by dry-mixing or by blending them in a syrup form of crystals, along with the probiotic strain(s) (if used), and the mixture is spray-dried or freeze-dried.

If a liquid composition is preferred, the homogenised mixture may be sterilised then aseptically filled into suitable containers or may be first filled into the containers and then retorted.

In therapeutic applications (treatment of growth delay for example), the sn-1(3) MAGs are administered in an amount sufficient to at least partially cure or arrest the symptoms of the disease/health problem and its complications. An amount adequate to accomplish this is defined as “a therapeutically effective dose”, i.e. an amount that prevents a deficiency, treats a deficiency or, more generally, reduces symptoms, manages progression of a deficiency or provides a nutritional, physiological, or medical benefit to an individual. Amounts effective for this purpose will depend on a number of factors known to those of skill in the art such as the severity of the deficiency, the weight and the general state of the infant/child.

In prophylactic applications (e.g. prevention of growth delay for example), the sn-1(3) MAGs are administered to the infant/child susceptible to or otherwise at risk of a particular disease/health problem in an amount that is sufficient to at least partially reduce the risk of developing a disease/problem. Such an amount is defined to be “a prophylactic effective dose”. Again, the precise amounts depend on a number of patient specific factors such as the infant/child's state of health and weight.

The nutritional compositions of the present invention are to be administered in an amount sufficient to provide the sn-1(3) MAGs in a therapeutically effective dose or a prophylactic effective dose.

Similarly, the amount of fatty acids in the composition of the present invention may be adjusted to the infant/child needs.

By way of example, the sn-1(3) monoacylglycerol may provide from 0.0001% to 100% of the energy of the nutritional composition, such as 0.0005-70%, or 0.001-50% or 0.005-40% or 0.01-20% or 0.1-10% of the energy of the nutritional composition. It may provide for example from 0.001 to 5% or from 0.005 to 2% or from 0.01 to 1% of the energy of the nutritional composition. It may also provide from 0.01 to 5%, or from 0.05 to 10%, or from 10 to 20%, or from 30 to 60%, or from 40-55% of the energy of the nutritional composition. In a particular example, it may provide 100% of the energy when the nutritional composition of the invention consists only of the sn-1(3) monoacylglycerol, for example in case of fat fortifier or supplement.

When the nutritional composition is an infant formula (e.g. a preterm infant formula, a starter infant formula, a follow-up or follow-on formula), a baby food, an infant cereal composition or a growing-up milk, it can comprise for example between 0.0001% and 30% of energy of sn-1(3) monoacylglycerols, such as 0.0005-20% of energy, or 0.001-15% of energy, or 0.001-10% of energy. It may be from 0.005% to 10%, or 0.01-7%, or 0.02-5% or 0.02-2% or 0.02-1% or 0.02-0.5% of energy.

When the nutritional composition is a fortifier such as a human milk fortifier, or a supplement, it can comprise for example between 0.0001% and 100% of energy of sn-1(3) monoacylglycerols, such as 0.0005-100%, 0.001-100% of energy. It may comprise low amounts such as from 0.005% to 10%, or 0.01-7%, or 0.02-5%, or 0.02-2%, or 0.02-1%, or 0.02-0.5% of energy. It may also comprises higher amounts such as from 25 to 100%, or 40-100%, 50-100%, 70-100%, 80-100% or 90-100%, or such as 10-90%, 20-80% or 40-60% of energy.

When the nutritional composition is supplement comprising sn-1(3) monoacylglycerols, it should be provided in an amount sufficient to achieve the desired effect in an individual. The daily dose of sn-1(3) monoacylglycerols is typically from 1 mg/Kg body weight/day to 20 g Kg body weight/day depending on the intended use.

The supplement may be in the form of tablets, capsules, pastilles or a liquid for example. The supplement may further contain protective hydrocolloids (such as gums, proteins, modified starches), binders, film forming agents, encapsulating agents/materials, wall/shell materials, matrix compounds, coatings, emulsifiers, surface active agents, solubilizing agents (oils, fats, waxes, lecithins etc.), adsorbents, carriers, fillers, co-compounds, dispersing agents, wetting agents, processing aids (solvents), flowing agents, taste masking agents, weighting agents, jellifying agents and gel forming agents. The supplement may also contain conventional pharmaceutical additives and adjuvants, excipients and diluents, including, but not limited to, water, gelatine of any origin, vegetable gums, lignin-sulfonate, talc, sugars, starch, gum arabic, vegetable oils, polyalkylene glycols, flavouring agents, preservatives, stabilizers, emulsifying agents, buffers, lubricants, colorants, wetting agents, fillers, and the like.

Further, the supplement may contain an organic or inorganic carrier material suitable for oral or parenteral administration as well as vitamins, minerals trace elements and other micronutrients in accordance with the recommendations of Government bodies such as the USRDA.

The nutritional composition according to the invention can be administered (or given, fed, eaten, ingested . . . ) to the infant/child at an age and for a period that depends on the needs.

In some embodiments the nutritional composition is used for the prevention of growth delay of an infant/child.

In some other embodiments the nutritional composition of the invention is for use in the treatment of growth delay of an infant/child, i.e. when the infant or the child (e.g. a young child) already suffers from growth delay.

In some other embodiments the nutritional composition of the invention is for use in the promotion of growth of an infant/child.

For example the composition can be given immediately after birth of the infants. The composition of the invention can also be given during the first week of life, or during the first 2 weeks of life, or during the first 3 weeks of life, or during the first month of life, or during the first 2 months of life, or during the first 3 months of life, or during the first 4 months of life, or during the first 6 months of life, or during the first 8 months of life, or during the first 10 months of life, or during the first year of life, or during the first two years of life of the infant/child or even more. In some other embodiments, the nutritional composition of the invention is not given immediately but few days, or few weeks, or few months after birth. This may be especially the case when the infant is premature or LBW, but not necessarily. This may be also the case when the nutritional composition is for use in children such as young children.

In one embodiment the nutritional composition of the invention is given to the infant or to the child (especially to young child), as a supplementary composition to the mother's milk. In one embodiment the composition is given to the infant or child as the sole or primary nutritional composition during at least one period of time, e.g. after the 1^{st}, 2^{nd} or 4^{th} month, during at least 1, 2, 4 or 6 months. In some embodiments the infant or child receives the mother's milk during at least the first 2 weeks, first 1, 2, 4, or 6 months. In one embodiment the composition of the invention is given to the infant or child after such period of mother's nutrition, or is given together with such period of mother's milk nutrition.

In one embodiment the nutritional composition of the invention is for use in an infant only during the first week, the first 2, 4 weeks, or the first 2 or 4 months of life.

In one embodiment the nutritional composition of the invention is a complete nutritional composition (fulfilling all or most of the nutritional needs of the subject). In another embodiment the nutrition composition is a supplement or a fortifier intended for example to supplement human milk or to supplement an infant formula, a follow-on or follow-up formula, or a growing-up milk.

The nutritional composition of the invention can be given for some days (1, 2, 3, 4, 5, 6 . . . ), or for some weeks (1, 2, 3, 4, 5, 6, 7, 8 or even more), or for some months (1, 2, 3, 4, 5, 6, 7, 8, 9, 10, 11 or even more), or for some years, depending on the needs.

In some particular embodiments, the nutritional composition of the invention is not directly administered to the infant or child but to the lactating mother, i.e. the composition will be indirectly administered to the infant via the lactating mother's breast milk. Similarly the nutritional composition of the invention can be given to the mother for some days (1, 2, 3, 4, 5, 6 . . . ), or for some weeks (1, 2, 3, 4, 5, 6, 7, 8 or even more), or for some months (1, 2, 3, 4, 5, 6, 7, 8, 9, 10, 11 or even more), or for some years, depending on the needs.

The present invention also relates to the use of sn-1(3) monoacylglycerols in the preparation of a nutritional composition for the prevention/treatment of growth delay and/or for the promotion of growth in an infant or in a child (like a young child).

The present invention also relates to the use of sn-1(3) monoacylglycerols in the preparation of a nutritional composition for providing nutrition (or a suitable nutrition) to an infant or to a child (like a young child).

The present invention also relates to a method for preventing and/or treating growth delay in an infant or a child (like a young child), said method comprising administering to said infant or child a nutritional composition comprising sn-1(3) monoacylglycerols. The present invention also relates to a method for promoting growth in an infant or a child (like a young child), said method comprising administering to said infant or child a nutritional composition comprising sn-1(3) monoacylglycerols.

The present invention also relates to a method for providing nutrition (or a suitable nutrition) to an infant or a child (like a young child), said method comprising administering to said infant or child a nutritional composition comprising sn-1(3) monoacylglycerols.

The different embodiments, details and examples previously described in the specification can similarly be applied to these uses and methods.

Further advantages and features of the present invention will be presented in the following Examples and Figures.

### EXAMPLES

The following examples illustrate some specific embodiments of the composition for use according to the present invention. The examples are given solely for the purpose of illustration and are not to be construed as limitations of the present invention, as many variations thereof are possible without departing from the spirit of the invention.

### Example 1

An example of the composition of an infant formula according to the present invention is given in the below table 1. This composition is given by way of illustration only.

TABLE 1
example of the composition of an infant formula (e.g. a preterm formula)
according to the present invention

| Nutrient | per 100 kcal | per litre |
|---|---|---|
| Energy (kcal) | 100 | 670 |
| Protein (g) | 1.83 | 12.3 |
| Fat (g) | 5.3 | 35.7 |
| Linoleic acid (g) | 0.79 | 5.3 |
| α-Linolenic acid (mg) | 101 | 675 |
| sn-1(3)MAG-DHA (g) | 0.022 | 0.15 |
| Lactose (g) | 11.2 | 74.7 |
| Minerals (g) | 0.37 | 2.5 |
| Na (mg) | 23 | 150 |
| K (mg) | 89 | 590 |
| Cl (mg) | 64 | 430 |
| Ca (mg) | 62 | 410 |
| P (mg) | 31 | 210 |
| Mg (mg) | 7 | 50 |
| Mn (μg) | 8 | 50 |
| Se (μg) | 2 | 13 |
| Vitamin A (μg RE) | 105 | 700 |
| Vitamin D (μg) | 1.5 | 10 |
| Vitamin E (mg TE) | 0.8 | 5.4 |
| Vitamin K1 (μg) | 8 | 54 |
| Vitamin C (mg) | 10 | 67 |
| Vitamin B1 (mg) | 0.07 | 0.47 |
| Vitamin B2 (mg) | 0.15 | 1.0 |
| Niacin (mg) | 1 | 6.7 |
| Vitamin B6 (mg) | 0.075 | 0.50 |
| Folic acid (μg) | 9 | 60 |
| Pantothenic acid (mg) | 0.45 | 3 |
| Vitamin B12 (μg) | 0.3 | 2 |
| Biotin (μg) | 2.2 | 15 |
| Choline (mg) | 10 | 67 |
| Fe (mg) | 1.2 | 8 |
| 1 (μg) | 15 | 100 |
| Cu (mg) | 0.06 | 0.4 |
| Zn (mg) | 0.75 | 5 |

### Example 2

The concept was tested in a lipid maldigestion or malabsorption rat model. The maldigestion or malabsorption condition was obtained using XENICAL® (ORLISTAT, tetrahydrolipstatin), a pancreatic and gastric lipases inhibitor. Rats were fed during 21 days with long-chain polyunsaturated fatty acid (LC-PUFA) supplements containing mainly eicosapentaenoic (EPA) acid. Fish oil was used as a source of triacylglycerols, and different EPA glycerides were evaluated: Vanillin acetal of 2-EPA (from Stepan Lipid Nutrition); 1,3 Diacetyl-2 EPA (from Stepan Lipid Nutrition) and Sn-1(3)-MAG-EPA (purchased from Cognis GmbH, Germany).

XENICAL® (ORLISTAT) was given at a level sufficient to decrease lipid absorption by 40%. A group receiving fish oil without XENICAL® (ORLISTAT) was used as a positive control. At different time intervals (D3, D7, D14 and D21), the fatty acid profiles of red blood cells and plasma lipids were determined. At the end of the experiment, the fatty acid profiles of different tissues were determined.

The main objective was to follow the level of EPA in red blood cells and plasma lipids. The main comparison evaluated was the difference in EPA level between the group receiving EPA glycerides such as EPA-containing sn-1(3) MAG, in combination with XENICAL® (ORLISTAT) and the positive control group (fish oil+XENICAL® (ORLISTAT)).

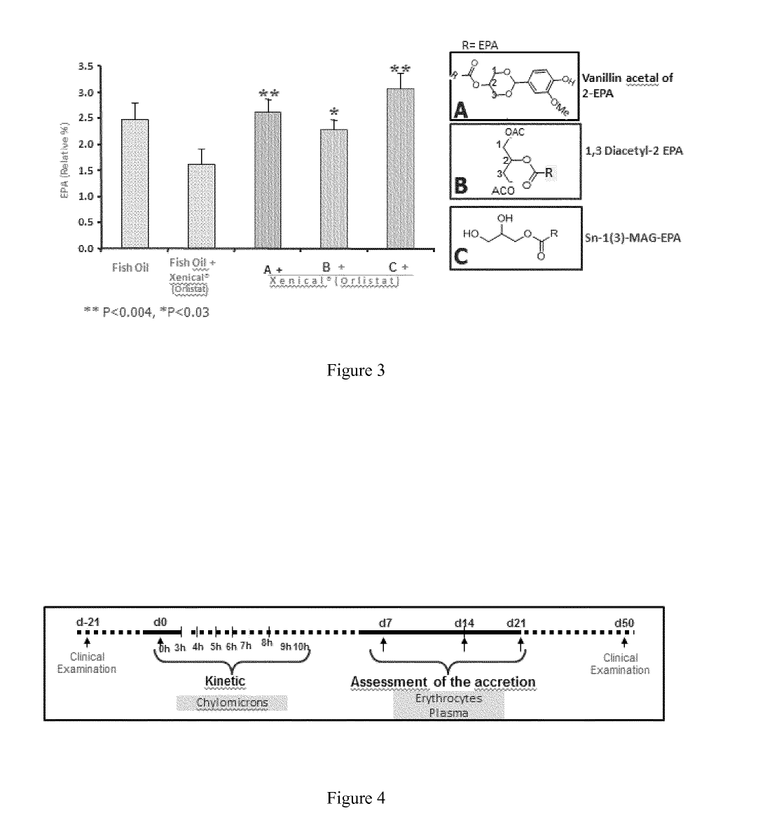

As an example, data obtained for EPA levels in red blood cell lipids at day 7 are reported in FIG. 3. The statistical evaluation revealed that the use of XENICAL® (ORLISTAT) decreases EPA incorporation in red blood cells (comparison between the group receiving fish oil in combination with XENICAL® (ORLISTAT) and the group receiving fish oil without XENICAL® (ORLISTAT)). This comparison corroborates the validity of the model. The level of EPA incorporated in red blood cells in animals receiving the sn-1(3) MAG that contained EPA is statistically higher that the fish oil+group receiving fish oil in combination with XENICAL® (ORLISTAT) (all P values lower that 0.05), and more surprisingly, even higher than the fish oil group.

This example clearly demonstrates that in conditions of lipid maldigestion or malabsorption, the incorporation of LC-PUFAs provided as triacylglycerols is reduced. However, when comparing groups A, B and C, it was surprisingly seen that if LC-PUFAs are provided as sn-1(3) MAG (Group C), the incorporation in tissue is improved, even in conditions of lipid maldigestion or malabsorption.

### Example 3

This clinical study compared the efficacy of sn-1(3) MAG and fish oil (TAG) in delivering EPA in humans under lipid maldigestion conditions induced by XENICAL® (ORLISTAT). The comparison was tested in volunteers treated with XENICAL® to induce lipid maldigestion or not treated with XENICAL® (ORLISTAT). The primary objective was to assess accretion of EPA in erythrocytes over 21 days when consumed as fish oil (TAG) or sn-1(3) MAG. The secondary objectives were to assess accretion of EPA in plasma over 21 days and also to assess the pharmacokinetics of EPA after an acute dose either in the form of sn-1(3) MAG or TAG (AUC in chylomicrons over 10 hours postprandial). See FIG. 4 that describes the clinical study timeline.

TABLE 2
Experimental Groups

| | | | | XENICAL ® | |
|---|---|---|---|---|---|
| | | | | (Orlistat) | |
| | Oil Type and | | | (120 mg) | |
| Group | number | | Total EPA | and number | |
| No. | per day | | (mg) | per day | |
| 1 (n = 10) | Fish oil | 3 | 504 | No | — |
| 2 (n = 10) | sn-1(3) MAG | 3 | 500 | No | — |
| 3 (n = 11) | Fish oil | 3 | 504 | Yes | 3 |
| 4 (n = 11) | sn-1(3) MAG | 3 | 500 | Yes | 3 |

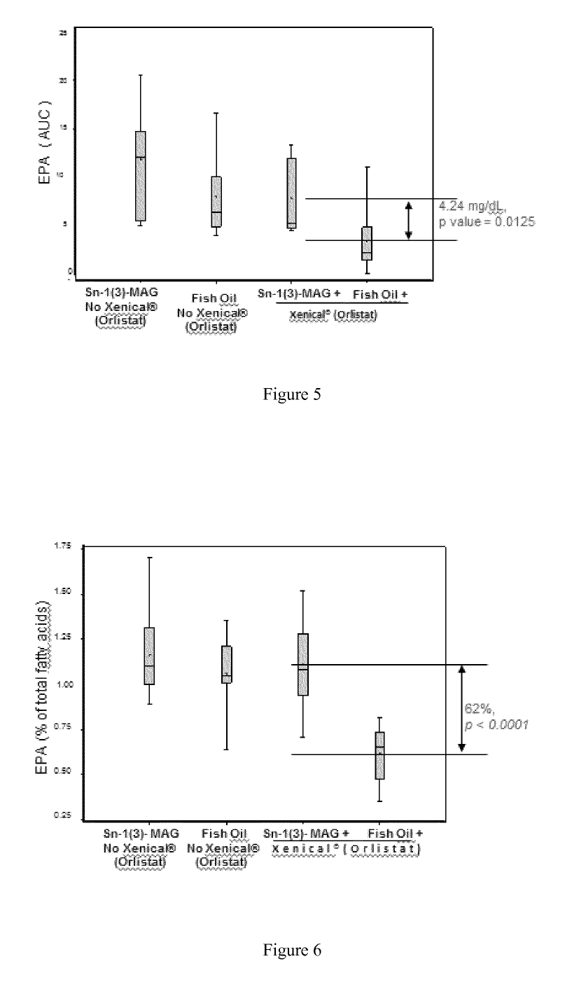

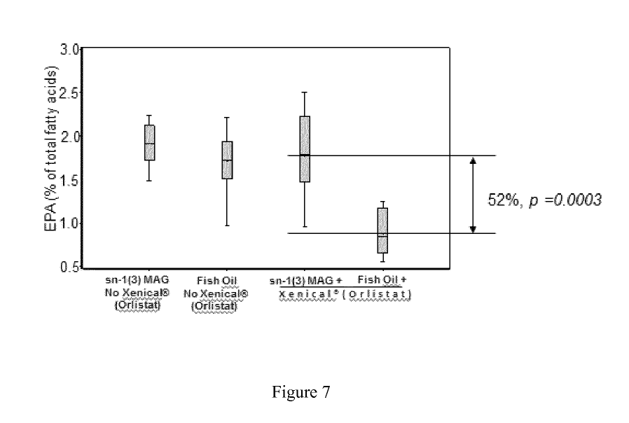

The pharmacokinetic results (FIG. 5) show that the acute effect from treatment with sn-1(3) MAG and XENICAL® (ORLISTAT) is statistically significant relative to treatment with fish oil and XENICAL® (ORLISTAT) (p=0.0125). The accretion of EPA in erythrocytes after 21 days (FIG. 6) shows that the chronic effect of treatment with sn-1(3) MAG and XENICAL® (ORLISTAT) is statistically significant, especially in comparison to treatment with fish oil and XENICAL® (ORLISTAT) (p=0.0001). The accretion of EPA in plasma after 21 days (FIG. 7) shows that the chronic effect of treatment with sn-1(3) MAG and XENICAL® (ORLISTAT) is statistically significant relative to treatment with fish oil and XENICAL® (ORLISTAT) (p=0.0003).

This clinical trial confirmed that, in human subjects treated with XENICAL® (ORLISTAT), sn-1(3) MAG is a better carrier for EPA than fish oil (TAG).

### Example 4

### In Vitro Digestion to Assess Lipidic Components Bioaccessibility.

Simulated or in vitro digestion is a model to be used to assess the stability of lipidic components such as fatty acids, liposoluble vitamins and carotenoids, during the digestive phases (oral, gastric and small intestinal) and the extent of partitioning of lipidic components into mixed bile salt micelle fraction (essential step for absorption of lipophiles). Partitioning of lipidic components into mixed bile salt micelle is also referred as “bioaccessibility” and expressed as efficiency of micellarization. In each step, the type of enzymes is adapted as needed (e.g malabsorption vs. control).

Briefly, samples will be adjusted and subjected to simulated digestion accordingly, to better reflect physiologic conditions in the gut. When having high starch content, the oral phase of digestion is included as well as addition of α-amylase. A basal salt solution containing NaCl, KCl and CaCl₂ is required for simulated gastric and small intestinal digestion. KCl is added as a second physiological salt besides NaCl and CaCl₂ are added for maximal activity of lipases.

The pH of gastric digestion is adjusted as well as for that of small intestinal digestion. Porcine pancreatic lipase, pancreatin and bile extract are added to facilitate lipid digestion. Finally, the micelle fraction is isolated from digesta by centrifugation and filtration of the collected aqueous fraction.

Extraction of Lipidic Components from Digesta Fraction Resulting from In Vitro Digestion.

Analytical techniques will be adapted, to identify and quantify lipidic components of interest and their digestion products. Briefly, aliquots of micelle fraction and digesta are mixed with THF:hexane after addition of a recovery standard. Mixing and centrifugation are followed by evaporation of solvents to finalize with lipidic components reconstituted in 1 mL of mobile phase for HPLC or UPLC analysis.

### Analysis and Quantification of Lipidic Components.

A method involving an alkaline hydrolysis treatment of samples with a liquid-liquid extraction, an Ultra Performance Liquid Chromatography (UPLC) separation, and fluorescence and UV-visible detection for quantification will be used to quantify different lipidic components.

1. A method for the prevention/treatment of growth delay and/or in the promotion of growth in an infant or a child comprising administering a nutritional composition comprising sn-1(3) monoacylglycerols to an individual in need of same.

2. Method according to claim 1, wherein the sn-1(3)-monoacylglycerol is selected from the group consisting of sn-1(3)-monohexadecanoylglycerol, sn-1(3)-monotetradecanoylglycerol, sn-1(3)-monoeicosatetraenoilglycerol, sn-1(3)-monooctadecanoylglycerol, sn-1(3)-monooctadecadienoylglycerol, sn-1(3)-monoeicosapentaenoylglycerol, sn-1(3)-mono docosahexaenoylglycerol, sn-1 (3)-monooctadecatrienoylglycerol, sn-1(3)-monooctadecatetraenoylglycerol, sn-1(3)-monoeicosatrienoylglycerol, sn-1(3)-monodocosapentaenoylglycerol, sn-1(3)-monosciadonylglycerol, sn-1(3)-monojuniperonylglycerol and combinations thereof.

3. Method according to claim 1, wherein the sn-1(3)-monoacylglycerol is selected from the group consisting of sn-1(3) monoeicosapentaenoylglycerol, sn-1(3)-mono docosahexaenoylglycerol, sn-1(3)-monoeicosatetraenoilglycerol, sn-1(3)-monooctadecadienoylglycerol, sn-1(3)-monooctadecatrienoylglycerol and combinations thereof.

4. Method according to claim 1, wherein the sn-1(3) monoacylglycerol provides from 0.0001% to 100% of the energy of the nutritional composition.

5. Method according to claim 1, wherein the sn-1(3) monoacylglycerols comprises at least one functional fatty acid.

6. Method according to claim 5, wherein the functional fatty acid is selected from the group consisting of tetradecanoic acid (myristic acid), hexadecanoic acid (palmitic acid), octadecanoic acid (stearic acid), eicosapentaenoic acid (EPA), docosahexaenoic acid (DHA), alpha-linolenic acid (ALA), linoleic acid (LA), arachidonic acid (ARA), stearidonic acid (SA), γ-linolenic acid (GLA), dihomo-γ-linolenic acid (DGLA), n-3 docosapentanenoic acid (DPA), sciadonic acid and juniperonic acid.

7. Method according to claim 1, wherein the sn-1(3) monoacylglycerols (MAG) comprise at least one of sn-1(3) MAG-EPA, sn-1(3) MAG-DHA, sn-1(3) MAG-LA, sn-1(3) MAG-ALA or sn-1(3) MAG-ARA.

8. Method according to claim 1, wherein growth refers to the size of the infant or child and/or to any organ or tissue development of the infant or child.

9. Method according to claim 1, wherein fat absorption in an infant or a child is improved.

10. Method according to claim 1 for use in the promotion of bone's growth and quality in an infant or a child.

11. Method according to claim 1 for use in the cognitive development, the motor and/or the behavioural development in an infant or a child.

12. Method according to claim 1 for use in the visual acuity development and/or the reduction of ROP risks in an infant or a child.

13. Method according to claim 1 for use in the lung development and/or the reduction of BPD risks in an infant or a child.

14. Method according to claim 1 for use in the cardiovascular system health and development in an infant or a child.

15. (canceled)

16. Method according to claim 1 wherein the infant or the child was born preterm and/or is small for gestational age (SGA) and/or has/had a low birth weight and/or is sick.

17. (canceled)

18. Method according to claim 1, wherein the nutritional composition is in a form selected from the group consisting of an infant formula, a starter infant formula, a follow-up or a follow-on infant formula, a growing-up milk, a baby food, an infant cereal composition, a fortifier and a supplement.

19. Method according to claim 1, wherein the nutritional composition comprises a protein source, a carbohydrate source and a lipid source.

20. A method for providing nutrition to an infant or a child comprising administering to the infant or child sn-1(3) monoacylglycerols in a nutritional composition.

21. Method according to claim 20, wherein the sn-1(3) monoacylglycerols is selected from the group consisting of sn-1(3)-monohexadecanoylglycerol, sn-1(3)-monotetradecanoylglycerol, sn-1(3)-monoeicosatetraenoilglycerol, sn-1(3)-monooctadecanoylglycerol, sn-1(3)-monooctadecadienoylglycerol, sn-1(3)-monoeicosapentaenoylglycerol, sn-1(3)-mono docosahexaenoylglycerol, sn-1(3)-monooctadecatrienoylglycerol, sn-1(3)-monooctadecatetraenoylglycerol, sn-1(3)-monoeicosatrienoylglycerol, sn-1(3)-monodocosapentaenoylglycerol, sn-1(3)-monosciadonylglycerol, sn-1(3)-monojuniperonylglycerol and combinations thereof.

22. Method according to claim 20 wherein the infant or child is preterm and/or low birth weight.
